(19) United States
(12) Patent Application Publication
(10) Pub. No.: US 2004/0097515 A1
(43) Pub. Date: May 20, 2004

(54) Antimicrobial drug reduced in effect on heart

(76) Inventors: Hisashi Takahashi, Tokyo (JP); Takehiro Hagiwara, Tokyo (JP); Isao Hayakawa, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/472,183

(22) PCT Filed: Mar. 27, 2002
(86) PCT No.: PCT/JP02/02969

(30) Foreign Application Priority Data
Mar. 27, 2001 (JP) 2001-88988

(51) Int. Cl. A61K 31/496; C 07D 4 1/04

(52) U.S. Cl. 514/252.13; 544/360

(57) ABSTRACT

An antimicrobial drug containing a compound represented by the following formula (I):

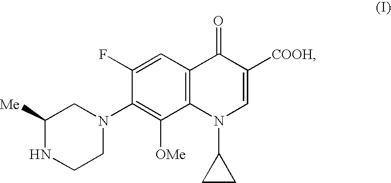

a salt of the compound, or a hydrate of the compound or the salt.

### TECHNICAL FIELD

The present invention relates to an antimicrobial drug and an antimicrobial drug product which exhibit enhanced safety (particularly, reduced cardiac toxicity).

### BACKGROUND ART

Gatifloxacin is a quinolone-based synthetic antimicrobial drug having the following structure:

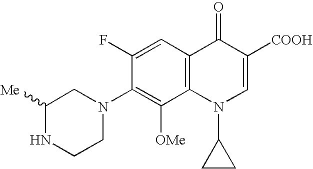

and is now under development in countries around the world (in fact, a particular form of the compound; i.e., a sesquihydrate of gatifloxacin, is under development (Japanese Patent Application Laid-Open (kokai) No. 62-252772)).

As is clear from the structure of gatifloxacin, the 3-methylpiperazine moiety of gatifloxacin, which is the substituent at position 7 of the quinolone skeleton, has an asymmetric carbon atom. Therefore, gatifloxacin has optical antipodes attributed to the piperazine substituent.

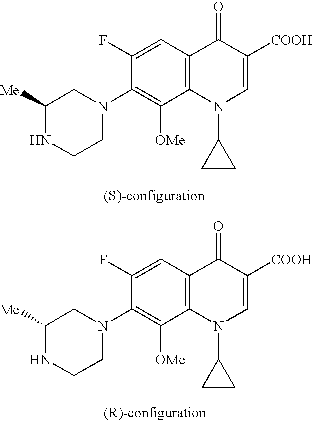

Specifically, gatifloxacin has an optical antipode having an (S) configuration at position 3 of the piperazine moiety, and an optical antipode having an (R) configuration at position 3 of the piperazine moiety [hereinafter, the antipode having the (S) configuration and the antipode having the (R) configuration will be referred to as “compound (I)” and “compound (II),” respectively]. As has been reported, these antipodes exhibit no great difference in antimicrobial activity and pharmacokinetic action in the body (Japanese Journal of Chemotherapy; 27-30, Volume 47, 1999; 112-123, Volume 47, 1999; and 124-130, Volume 47, 1999).

Regarding quinolone-based synthetic antimicrobial drugs, many compounds have been found to exhibit high antimicrobial activity (i.e., main pharmacological action), and on the other hand, many compounds have been found to involve problems in terms of safety (e.g., high toxicity). Therefore, demand has arisen for an antimicrobial drug exhibiting low toxicity and higher safety.

The present inventors have performed extensive studies, and as a result have found that, surprisingly, as compared with the (R)-antipode of gatifloxacin, the (S)-antipode of gatifloxacin is a compound exhibiting significantly lower effect of prolonging APD (hereinafter the effect may be referred to as “APD prolonging effect”), which effect indicates cardiac toxicity. That is, the present inventors have found that the (S)-antipode of gatifloxacin is a compound which exhibits reduced cardiac toxicity and is more suitable for use as a drug. The present invention has been accomplished on the basis of this finding.

Accordingly, the present invention provides an antimicrobial drug comprising a compound represented by the following formula (I):

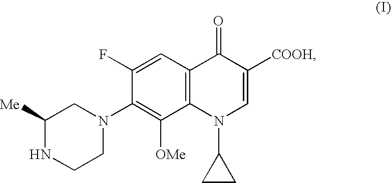

a salt of the compound, or a hydrate of the compound or the salt. The present invention is directed to an antimicrobial drug exhibiting, particularly, reduced cardiac toxicity.

The present invention also provides an antimicrobial drug composition comprising a compound represented by formula (I), a salt of the compound, or a hydrate of the compound or the salt, and a pharmacologically acceptable carrier.

The present invention also provides use of a compound represented by formula (I), a salt of the compound, or a hydrate of the compound or the salt, for producing an antimicrobial drug.

The present invention also provides a method for treating infectious diseases of animals including human, which comprises administering, to a subject in need thereof, an effective dose of a compound represented by formula (I), a salt of the compound, or a salt of the compound or the salt.

### BRIEF DESCRIPTION OF THE DRAWINGS

FIG. 1 shows the effect of compound (I), compound (II), or gatifloxacin on the myocardial action potential duration (APD₉₀) of a guinea pig right ventricular myocardium sample; and

FIG. 2 shows the effect of compound (I), compound (II), or gatifloxacin on APD₉₀ of a guinea pig right ventricular myocardium sample after the sample is exposed to the compound (100 μM) for 60 minutes.

### BEST MODE FOR CARRYING OUT THE INVENTION

As described below, the present inventors performed studies on the APD prolonging effect—which is one of various effects on the heart—of gatifloxacin, compound (I) (i.e., one of the antipodes of gatifloxacin), and compound (II) (i.e., the other antipode). As a result, the present inventors found that compound (I) exhibits significantly lower APD prolonging effect, and thus has more preferred characteristics as a drug.

APD Prolonging Effect of Compounds (I) and (II)

Slc: Hartley male guinea pigs (body weight: 322 to 667 g each) were employed as subjects.

Each of compound (I), compound (II), and gatifloxacin was dissolved in an aqueous 1% lactic acid solution, to thereby prepare a 10 mM solution. The resultant solution was employed as a drug solution.

Measurement of action potential was performed as follows. Specifically, the heart was quickly removed from each of the guinea pigs, and in a nutrition solution (Krebs-Henseleit) aerated with 95% O₂+5% CO₂ at room temperature, the right ventricular free wall was dissected to thereby prepare a myocardium sample. By use of an injection needle and platinum bipolar electrodes for electric stimulation, the sample was horizontally fixed onto a silicon block within a 20 ml organ bath (maintained at 36.5±0.5° C.) perfused with the nutrition solution aerated with 95% O₂+5% CO₂. During the test, the sample was electrically stimulated at a frequency of 1 Hz by means of rectangular pulses (voltage: about 1.3 times the threshold at which contraction of the sample is initiated, duration: 1 msec) provided from an electrostimulator via the platinum bipolar electrodes. The action potential of the cells was measured via glass microelectrodes (electrode resistance: 5 to 30 MΩ) which were filled with a 3 M KCl solution and connected to an amplifier for microelectrodes, and the action potential was recorded and analyzed by use of a computer. After electrical stimulation of the sample was continued for one hour or more, the action potential was measured before administration of the drug solution, and 10, 20, 30, 40, 50, and 60 minutes after administration of the drug solution. At each point in time, the action potential was measured and recorded in triplicate, and the average of the measured potentials was taken as the value for that point in time.

The drug solution (200 μL) was added to the 20 mL organ bath by use of a micropipette (final concentration of the drug solution in the bath: 100 μM). On the basis of the thus-measured action potential waveform, resting membrane potential (RMP), overshoot (OS), action potential amplitude (APA), action potential durations at 20%, 50%, and 90% repolarization (APD₂₀, APD₅₀, and APD₉₀), and maximum rising velocity (Vmax) were obtained.

In each of the groups, the paired t-test was performed on the initial value of APD₉₀ and on the action potential measured at each of the aforementioned points in time. Also, time-course variance analysis was performed, and the action potential after the 60 minute-exposure of the sample to the drug solution was subjected to the Tukey test, to thereby perform comparison between the groups. The level of significance was set at 5%, two-sided.

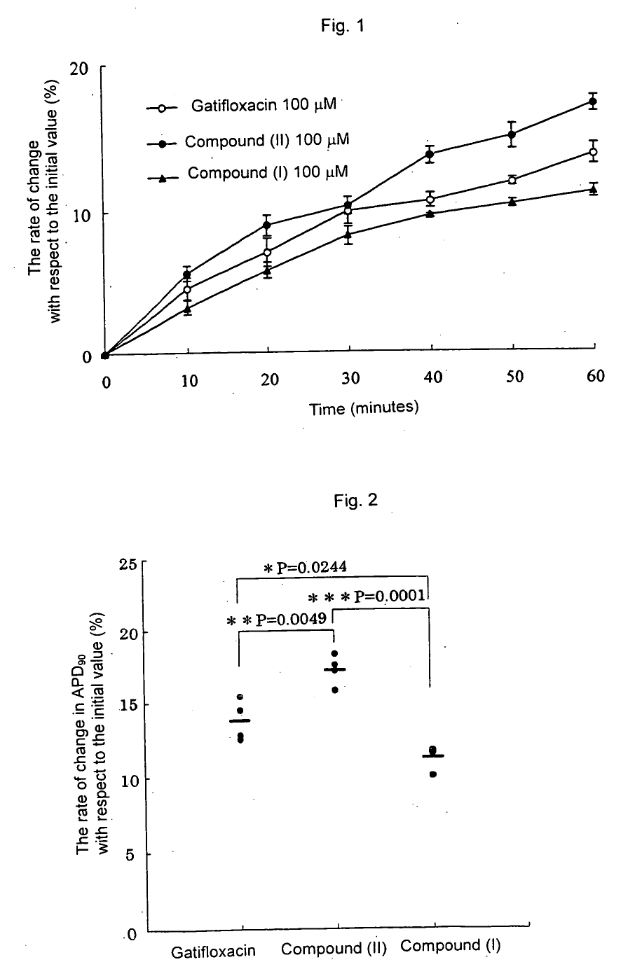

The results are described below. When the sample is exposed to 100 μM of gatifloxacin, compound (I), or compound (II) for 10 minutes or more, myocardial action potential duration is significantly prolonged [Table 1 (the results of the paired t-test), FIG. 1].

TABLE 1

| | | Time serial Number | | | | | | |
|---|---|---|---|---|---|---|---|---|
| | | 1 | 2 | 3 | 4 | 5 | 6 | 7 |
| | | Time | | | | | | |
| | | 0 | 10 | 20 | 30 | 40 | 50 | 60 |
| Gatifloxacin | Number of samples | 6 | 5 | 5 | 6 | 4 | 3 | 4 |
| | Average | 174.8600 | 180.6120 | 184.9200 | 192.2467 | 190.8950 | 201.5533 | 196.4925 |
| | Standard deviation | 16.3837 | 20.0346 | 19.5122 | 19.0006 | 20.6542 | 17.1345 | 22.4213 |
| | p value | — | 0.0059 | 0.0026 | 0.0002 | 0.0001 | 0.0009 | 0.0007 |
| | Mark | — | ** | ** | *** | *** | *** | *** |
| Compound (II) | Number of samples | 6 | 6 | 6 | 6 | 3 | 4 | 4 |
| | Average | 176.1567 | 185.9917 | 191.9500 | 194.2750 | 205.5900 | 203.1350 | 206.9400 |
| | Standard deviation | 15.3600 | 15.9409 | 17.1708 | 17.5182 | 8.0703 | 11.6771 | 11.2973 |
| | p value | — | 0.0002 | 0.0001 | 0.0000 | 0.0009 | 0.0001 | 0.0000 |
| | Mark | — | *** | *** | *** | *** | *** | *** |
| Compound (I) | Number of samples | 6 | 6 | 6 | 6 | 4 | 4 | 4 |
| | Average | 170.3200 | 175.7500 | 180.2217 | 184.2600 | 190.6275 | 192.0675 | 193.5025 |
| | Standard deviation | 19.8127 | 19.9781 | 21.2261 | 21.4606 | 26.6749 | 27.5343 | 27.1964 |
| | p value | — | 0.0009 | 0.0003 | 0.0001 | 0.0006 | 0.0014 | 0.0010 |
| | Mark | — | *** | *** | *** | *** | ** | ** |

The results of time-course variance analysis (Table 2) show that a significant difference is observed between the groups, but interaction between the groups and time is not observed. Thus, the groups show no difference in response pattern, but show a significant difference in strength of APD prolonging effect (the rate of change in APD₉₀ with respect to the initial value). At each of the measured points, the relative strength of APD prolonging effect is as follows: compound (II)>gatifloxacin>compound (I).

TABLE 2

| | Degree of | Average sum | | | |
|---|---|---|---|---|---|
| Factors | freedom | of squares | F statistic | p value | Mark |
| Group | 2 | 53.0065 | 8.2424 | 0.0038 | ** |
| Intersubject error | 15 | 6.4309 | 6.8921 | 0.0000 | *** |
| Time | 5 | 167.8185 | 179.8540 | 0.0000 | *** |
| Interaction | 10 | 0.8872 | 0.9508 | 0.4960 | n.s. |
| between group | | | | | |
| and time | | | | | |
| Intrasubject error | 53 | 0.9331 | | | |

When the Tukey test was performed after the 60 minute-exposure of the sample to the drug solution, a statistically significant difference was observed between the three compounds (compound (II)>gatifloxacin>compound (I)) (Table 3, FIG. 2).

Although the above data would be considered to lead to a conclusion that also compound (I) has the effect of widening the QT interval in an electrocardiogram, the strength of the prolonging effect of compound (I) was found to be lower than that of the prolonging effect of gatifloxacin or compound (II), which is the antipode of compound (I), and thus compound (I) was confirmed to be the safest compound.

TABLE 3

| | | Number of | | Standard | Standard | | | |
|---|---|---|---|---|---|---|---|---|
| Group | Dose | samples | Average | deviation | error | Gatifloxacin | Compound (II) | Compound (I) |
| Gatifloxacin | 100 | 4 | 13.7789 | 1.4018 | 0.7009 | — | −4.3186 | 3.2522 |
| Compound (II) | 100 | 4 | 17.1760 | 1.0544 | 0.5272 | 0.0049 ** | — | 7.5707 |
| Compound (I) | 100 | 4 | 11.2206 | 0.7974 | 0.3987 | 0.0244 * | 0.0001 *** | — |

Test results . . .
n.s.: no significant difference
* p < 0.05
** p < 0.01
*** p < 0.001

When compound (I) is used as a drug for human, the daily dose of the compound for an adult falls within a range of 50 mg to 1 g, preferably 100 mg to 500 mg.

When compound (I) is used as a drug for animals, the dose of the compound varies depending on the purpose of administration (treatment or prevention), the species and size of an animal to be treated, the species of the infected pathogenic bacterium, and the degree of the infection. The daily dose per kg body weight of the animal typically falls within a range of 1 mg to 200 mg, preferably 5 mg to 100 mg.

The daily dose is administered once a day or 2 to 4 times a day in a divided manner. If necessary, the daily dose may exceed the above range.

Compound (I) is active against a variety of microorganisms which cause various infectious diseases, and therefore the compound can be used for treating, preventing or alleviating diseases caused by these pathogens.

Examples of bacteria or bacteria-like microorganisms against which compound (I) is effective include the genus Staphylococcus, Streptococcus pyogenes, hemolytic streptococcus, Enterococcus, Pneumococcus, the genus Peptostreptococcus, Neisseria gonorrhoeae, Escherichia coli, the genus Citrobacter, the genus Shigella, Klebsiella pneumoniae, the genus Enterobacter, the genus Serratia, the genus Proteus, Pseudomonas aeruginosa, Haemophilus influenzae, the genus Acinetobacter, the genus Campylobacter, and Chlamydia trachomatis.

Examples of diseases caused by these pathogens include folliculitis, furuncle, carbuncle, erysipelas, phlegmon, lymphangitis (lymphadenitis), felon, subcutaneous abscess, hidradenitis, acne conglobata, infectious atheroma, perianal abscess, mastitis, superficial secondary infections such as traumatic injury, burn, and operative wound, pharyngolaryngitis, acute bronchitis, tonsillitis, chronic bronchitis, bronchiectasis, diffuse panbronchiolitis, secondary infection of chronic respiratory disease, pneumonia, pyelonephritis, cystitis, prostatitis, epididymitis, gonococcal urethritis, non-gonococcal urethritis, cholecystitis, cholangitis, bacillary dysentery, enteritis, uterine adnexitis, intrauterine infection, bartholinitis, blepharitis, hordeolum, dacryocystitis, tarsadenitis, corneal ulcer, otitis media, sinusitis, periodontitis, pericoronitis, jaw inflammation, peritonitis, endocarditis, sepsis, meningitis, and skin infection.

Compound (I) is also effective against various microorganisms which cause infectious diseases of animals, such as the genus Escherichia, the genus Salmonella, the genus Pasteurella, the genus Haemophilus, the genus Bordetella, the genus Staphylococcus, and the genus Mycoplasma.

Specific examples of such diseases include colibacillosis, pullorum, avian paratyphoid, fowl cholera, infectious coryza, staphylococcosis, and Mycoplasma infection in the case of birds; colibacillosis, salmonellosis, pasteurellosis, Haemophilus infection, atrophic rhinitis, exudative epidermitis, and Mycoplasma infection in the case of pigs; colibacillosis, salmonellosis, hemorrhagic sepsis, Mycoplasma infection, bovine pleuropneumonia, and mastitis in the case of cattle; colisepsis, Salmonella infection, hemorrhagic sepsis, uterine empyema, and cystitis in the case of dogs; and exudative pleurisy, cystitis, chronic rhinitis, Haemophilus infection, kitten diarrhea, and Mycoplasma infection in the case of cats.

The physical form of an antimicrobial drug or antimicrobial drug composition containing compound (I) can be appropriately chosen in accordance with the manner of administration, and can be prepared by means of any of generally employed various drug preparation methods. Examples of the physical form of the antimicrobial drug product predominantly containing the compound of the present invention include oral drugs such as a tablet, a powder, a granule, a capsule, a solution, a syrup, an elixir, and an oily or aqueous suspension. In the case of preparation of an injection product, the injection product may contain a stabilizer, a preservative, or a dissolving aid. Alternatively, a solution which may contain such an auxiliary agent may be stored in a container and then formed into, by means of freeze-drying or a similar technique, a solid drug product which is prepared into an injection upon use. One dose of the drug product may be placed in a single container, or alternatively, multiple doses thereof may be placed in a single container.

Examples of external-use drug products include a solution, a suspension, an emulsion, an ointment, a gel, a cream, a lotion, and a spray.

In the case of preparation of a solid drug product, the drug product may contain, in addition to the active compound of the present invention, a pharmacologically acceptable carrier (additive). For example, according to needs, the active compound may be mixed with an appropriately selected additive, such as a filler, an extender, a binder, a disintegrating agent, a dissolution promoter, a humectant, or a lubricant, to thereby prepare a solid drug product. Examples of liquid drug products include a solution, a suspension, and an emulsion. Such a liquid drug product may contain an additive; for example, a suspending agent or an emulsifying agent.

Examples of the method for administering compound (I) to an animal include a method in which compound (I) is directly administered to the animal through an oral route, or a compound (I) is mixed with a feed and the resultant mixture is orally administered to the animal; a method in which a solution prepared from compound (I) is directly administered to the animal through an oral route, or the solution is added to drinking water or a feed and the resultant mixture is orally administered to the animal; and a method in which compound (I) is administered to the animal through injection. When the compound of the present invention is administered to an animal, the compound can be prepared into an appropriate drug product, such as a powder, a fine granule, a soluble powder, a syrup, a solution, or an injection, by means of a technique which is generally employed in the art.

Formulation examples of the drug product are described below.

### PREPARATION EXAMPLE 1

### Capsule

| | |
|---|---|
| Compound (I) | 100.0 mg |
| Corn starch | 23.0 mg |
| CMC calcium | 22.5 mg |
| Hydroxymethyl cellulose | 3.0 mg |
| Magnesium stearate | 1.5 mg |
| Total | 150.0 mg |

### PREPARATION EXAMPLE 2

### Solution

| | | |
|---|---|---|
| Compound (I) | 1-10 | g |
| Acetic acid or sodium hydroxide | 0.5-2 | g |
| Ethyl p-hydroxybenzoate | 0.1 | g |
| Purified water | 88.9-98.4 | g |
| Total | 100 | g |

### PREPARATION EXAMPLE 3

### Powder to be Mixed with Feed

| | | |
|---|---|---|
| Compound (I) | 1-10 | g |
| Corn starch | 98.5-89.5 | g |
| Light silicic acid anhydride | 0.5 | g |
| Total | 100 | g |

### PRODUCTION EXAMPLE 1

### 1-Cyclopropyl-6-fluoro-1,4-dihydro-8-methoxy-7-{3-(S)-methylpiperazin-1-yl}-4-oxoquinoline-3-carboxylic acid: compound (I)

1-Cyclopropyl-6,7-difluoro-1,4-dihydro-8-methoxy-4-oxoquinoline-3-carboxylic acid-BF₂ chelate (1.50 g, 4.37 mmol) was dissolved in dry dimethyl sulfoxide (22 ml), and subsequently (S)-(+)-2-methylpiperazine (657 mg, 6.56 mmol) was added to the resultant solution. The resultant mixture was stirred at room temperature for 15 hours, the resultant reaction mixture was added to water (400 ml), and the solid that precipitated was collected from the mixture through filtration and then washed with water. To the thus-obtained solid were added a solvent mixture of methanol and water (4:1) (130 ml) and triethylamine (1.83 ml, 13.1 mmol), and the resultant mixture was refluxed under heating for two hours. After having been left to cool, the resultant reaction mixture was concentrated under reduced pressure. The resultant residue was dissolved in an aqueous 1 N sodium hydroxide solution (40 ml), and then washed with dichloromethane (50 ml×3). The pH of the resultant basic aqueous solution was adjusted to 7.4 by use of concentrated hydrochloric acid and 1 N hydrochloric acid, and the resultant solution was subjected to extraction by use of chloroform (100 ml×3). The resultant extract was dried over anhydrous sodium sulfate, and subsequently the solvent was removed through evaporation under reduced pressure. The resultant residue was purified through recrystallization from ethanol, and then dried under reduced pressure, to thereby yield the title compound (652 mg (40%)) as yellow crystals.

¹H-NMR (400 MHz, 0.1N-NaOD) δ: 0.90-0.95 (2H, m), 1.07 (3H, d, J=5.86 Hz), 1.11-1.14 (2H, m), 2.86-3.00 (4H, m), 3.193.33 (3H, m), 3.75 (3H, s), 4.05-4.11 (1H, m), 7.70 (1H, d, J=12.69 Hz), 8.54 (1H, s).

IR (KBr disk): 3444, 2988, 2844, 1730, 1618, 1508, 1396, 1318, 1210 cm⁻¹

Melting point: 189-192° C.

Elementary analysis: C₁₉H₂₂FN₃O₄.0.5H₂O Calculated: C, 59.37; H, 6.03; N, 10.93. Found: C, 59.26; H, 6.12; N, 10.62.

Specific rotation: [α]_{D}²⁵−35.3° (c=1.015, 0.1 N NaOH).

The antimicrobial activity of the thus-obtained compound was measured by means of the standard method of Japanese Society of Chemotherapy. The results are shown in Table 4.

TABLE 4

| | |
|---|---|
| E. Coli, NIHJ | ≦0.003 |
| P. vulgaris 08601 | 0.006 |
| S. marcescens, 101000 | 0.10 |
| P. aeruginosa, 32104 | 0.20 |
| P. aeruginosa, 32121 | 0.10 |
| S. aureus, FDA 209-P | 0.05 |
| S. epidermidis, 56500 | 0.20 |
| E. faecalis, 19433 | 0.39 |

### PRODUCTION EXAMPLE 2

### 1-Cyclopropyl-6-fluoro-1,4-dihydro-8-methoxy-7-{3-(R)-methylpiperazin-1-yl}-4-oxoquinoline-3-carboxylic acid: compound (II)

1-Cyclopropyl-6,7-difluoro-1,4-dihydro-8-methoxy-4-oxoquinoline-3-carboxylic acid-BF₂ chelate (1.50 g, 4.37 mmol) was dissolved in dry dimethyl sulfoxide (22 ml), and subsequently (R)-(+)-2-methylpiperazine (736 mg, 7.35 mmol) was added to the resultant solution. The resultant mixture was stirred at room temperature for 15 hours, the resultant reaction mixture was added to water (400 ml), and the solid that precipitated was collected from the mixture through filtration and then washed with water. To the thus-obtained solid were added a solvent mixture of methanol and water (4:1) (130 ml) and triethylamine (1.83 ml, 13.1 mmol), and the resultant mixture was refluxed under heating for two hours. After having been left to cool, the resultant reaction mixture was concentrated under reduced pressure. The resultant residue was dissolved in an aqueous 1 N sodium hydroxide solution (40 ml), and then washed with dichloromethane (50 ml×3). The pH of the resultant basic aqueous solution was adjusted to 7.4 by use of concentrated hydrochloric acid and 1 N hydrochloric acid, and the resultant solution was subjected to extraction by use of chloroform (100 ml×3). The resultant extract was dried over anhydrous sodium sulfate, and subsequently the solvent was removed through evaporation under reduced pressure. The resultant residue was purified through recrystallization from ethanol, and then dried under reduced pressure, to thereby yield the title compound (984 mg (54%)) as yellow crystals.

¹H-NMR (400 MHz, 0.1N-NaOD) δ: 0.90-0.95 (2H, m), 1.07 (3H, d, J=5.86 Hz), 1.11-1.14 (2H, m), 2.86-3.00 (4H, m), 3.19-3.33 (3H, m), 3.75 (3H, s), 4.05-4.11 (1H, m), 7.70 (1H, d, J=12.69 Hz), 8.54 (1H, s).

IR (KBr disk): 3444, 2988, 2844, 1730, 1618, 1508, 1396, 1318, 1210 cm⁻¹

Melting point: 187-189° C.

Elementary analysis: C₁₉H₂₂FN₃O₄.0.5H₂O Calculated: C, 59.37; H, 6.03; N, 10.93. Found: C, 58.99; H, 6.13; N, 10.70.

Specific rotation: [α]_{D}²⁵+30.2° (c=1.055, 0.1 N NaOH).

Gatifloxacin was produced through the method described in Japanese Patent Application Laid-Open (kokai) No. 62-252772.

### INDUSTRIAL APPLICABILITY

Compound (I) exhibits reduced cardiac toxicity and has preferred characteristics as a drug. A drug product containing the compound is useful as an antimicrobial drug exhibiting reduced cardiac toxicity.

1. An antimicrobial drug comprising a compound represented by the following formula (I): 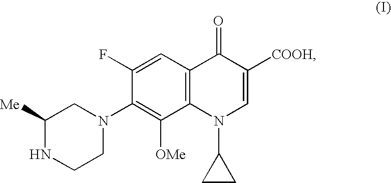
a salt of the compound, or a hydrate of the compound or the salt.

2. An antimicrobial drug composition comprising a compound represented by the following formula (I): 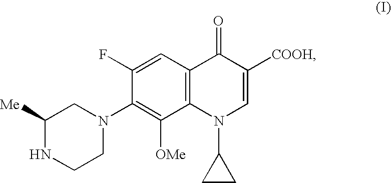
a salt of the compound, or a hydrate of the compound or the salt, and a pharmacologically acceptable carrier.

3. Use of a compound represented by the following formula (I): 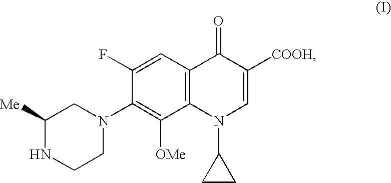
a salt of the compound, or a hydrate of the compound or the salt, for producing an antimicrobial drug.

4. A method for treating infectious diseases of animals including human, which comprises administering, to a subject in need thereof, an effective dose of a compound represented by the following formula (I): 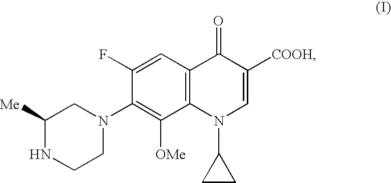
a salt of the compound, or a hydrate of the compound or the salt.
